(19) United States
(12) Patent Application Publication
(10) Pub. No.: US 2020/0214620 A1
(43) Pub. Date: Jul. 9, 2020

(54) METHOD AND APPARATUS FOR SKIN ASSESSMENT

(71) Applicant: Elizabeth Maureen Gallagher, Colorado Springs, CO (US)

(72) Inventor: Elizabeth Maureen Gallagher, Colorado Springs, CO (US)

(21) Appl. No.: 16/389,264

(22) Filed: Apr. 19, 2019

Related U.S. Application Data
(60) Provisional application No. 62/917,937, filed on Jan. 8, 2019.

(51) Int. Cl. A61B 5/00

(52) U.S. Cl. CPC A61B 5/444; A61B 5/447; A61B 5/445; A61B 5/6892; A61B 5/0059; A61B 2562/0247; A61B 5/6843

(57) ABSTRACT

An apparatus is disclosed. The apparatus has a skin assessment member. The skin assessment member is transparent. The skin assessment member is a single, integral member. The skin assessment member is nonporous. A length and a width of the skin assessment member are each between 20 times and 80 times larger than a thickness of the skin assessment member. A density of a material forming the skin assessment member is between 0.75 g/cm³ and 2.2 g/cm³. The skin assessment member includes a plurality of markings that are disposed flush with a flat surface of the skin assessment member or fully embedded within the skin assessment member.

### RELATED APPLICATIONS

This application claims the benefit of U.S. Provisional Patent Application Ser. No. 62/917,937 filed Jan.8, 2019, which is hereby incorporated by reference in its entirety.

### TECHNICAL FIELD

The present disclosure generally relates to a method and apparatus for assessment, and more particularly to a method and apparatus for skin assessment.

### BACKGROUND

Medical professionals such as nurses do multiple skin assessments daily. In home settings, family members may act as caretakers to also monitor elderly or immobilized family members to prevent and treat new injuries. These types of skin assessments often include blanch tests.

One conventional technique for conducting blanch tests involves a medical professional or individual performing finger blanch testing, where finger pressure is briefly applied to the skin of a patient by a practitioner's finger and then removed. However, the results and effectiveness of finger blanch testing are often variable based on the knowledge and expertise of the practitioner. In the case of inexperienced or untrained practitioners or laypeople, finger blanch testing may produce misleading or incorrect results.

U.S. Pat. No. 6,631,288 B1 (the '288 patent, directed to “Skin Evaluation Apparatus”) discloses one conventional technique for skin assessment. The '288 patent discloses the use of LED lighting and data interpretation, among other things, to make skin assessments. However, the '288 patent includes multiple complex parts that apparently make it cost-prohibitive and involves too much technical expertise for general and/or widespread use in hospital or home settings.

U.S. Pat. No. 7,155,273 B2 (the '273 patent, directed to “Blanching Response Pressure Sore Detector Apparatus and Method”) discloses another conventional technique for skin assessment. The '273 patent, however, involves a relatively complex system with multiple moving parts that would be relatively difficult to clean and cost-prohibitive for widespread use in hospital or home settings. Other conventional diascopy tools are similarly complex, cost-prohibitive, and/or not able to be readily available to users for widespread use.

The exemplary disclosed system and method are directed to overcoming one or more of the shortcomings set forth above and/or other deficiencies in existing technology.

### SUMMARY OF THE DISCLOSURE

In one exemplary aspect, the present disclosure is directed to an apparatus. The apparatus includes a skin assessment member. The skin assessment member is transparent. The skin assessment member is a single, integral member. The skin assessment member is nonporous. A length and a width of the skin assessment member are each between 20 times and 80 times larger than a thickness of the skin assessment member. A density of a material forming the skin assessment member is between 0.75 g/cm³ and 2.2 g/cm³. The skin assessment member includes a plurality of markings that are disposed flush with a flat surface of the skin assessment member or fully embedded within the skin assessment member.

In another aspect, the present disclosure is directed to method. The method includes providing an apparatus including a skin assessment member. The skin assessment member is transparent. The skin assessment member is a single, integral member. The skin assessment member is nonporous. The skin assessment member includes a plurality of markings that are disposed flush with a flat surface of the skin assessment member or fully embedded within the skin assessment member. The flat surface is part of a smooth exterior surface of the skin assessment member. The method also includes cleaning the smooth exterior surface, and applying constant pressure with the skin assessment member against a skin area of a patient. The plurality of markings are disposed over a wound or a skin condition of the skin area.

### BRIEF DESCRIPTION OF THE DRAWINGS

FIG. 1 depicts a front perspective view of an exemplary apparatus of the present invention;

FIG. 2 depicts a perspective view of an exemplary apparatus of the present invention;

FIG. 3 depicts a front perspective view of an exemplary apparatus of the present invention;

FIG. 4 depicts a front perspective view of an exemplary apparatus of the present invention;

FIG. 5 depicts a front perspective view of an exemplary apparatus of the present invention; and

FIG. 6 depicts a front perspective view of an exemplary apparatus of the present invention.

### DETAILED DESCRIPTION AND INDUSTRIAL APPLICABILITY

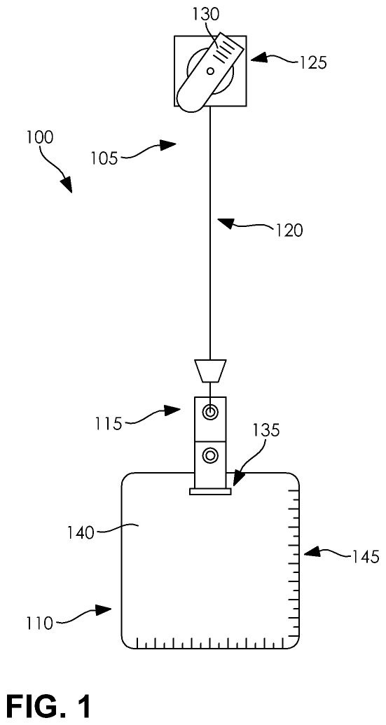

FIG. 1 illustrates an exemplary assembly 100 for skin assessment. Assembly 100 may include an attachment assembly 105 and an assessment device 110. Assessment device 110 may be attached to attachment assembly 105.

The exemplary disclosed assembly may be used in the field of non-invasive skin assessment. For example, assembly 100 may be a non-invasive skin assessment assembly and device. For example, assessment device 110 may be a skin assessment diascope and measurement tool. The exemplary disclosed assembly and assessment device may be a tool used by medical practitioners such as nurses and/or other users (e.g., laypeople) to monitor, prevent, and/or measure skin injuries, rashes, and other areas of concern of a human patient or animal. For example, the exemplary disclosed assembly may be a re-usable, non-invasive tool for rapidly assessing, measuring, and/or differentiating tissue damage of a patient or animal.

In at least some exemplary embodiments, the exemplary disclosed assembly and assessment device may be a skin assessment diascope and measurement tool that may be reusable. For example, assembly 100 may serve as an objective assessment tool, which may be cost-effective and affordable for widespread use by medical practitioners, laypeople, and any other suitable users. Assembly 100 may be used for the daily assessment and measurement of skin injury and other areas of concern. Assembly 100 may assist a user in diagnosing (e.g., identifying) certain skin rashes and other conditions that may be deemed medical emergencies by medical personnel. Users may be quickly trained and become rapidly efficient in the use and operation of assessment device 110, and may involve relatively little training costs and training time.

In at least some exemplary embodiments, the exemplary disclosed assembly and assessment device may be a lightweight and easily portable assembly and device (e.g., may be easily carried, stored, and/or worn by users). The exemplary disclosed assembly and assessment device may be a compact and substantially flat device (e.g., a non-bulky device).

In at least some exemplary embodiments, the exemplary disclosed assembly and assessment device may be a skin assessment diascope and measurement tool, which may be used as an effective assessment and monitoring tool in both medical and non-medical settings. Users may become proficient with the exemplary disclosed assembly and assessment device (e.g., diascope) with minimal training.

Components of assembly 100 may be formed from any suitable material for an assessment device and/or attachment assembly such as, for example, lightweight material. For example, components of assembly 100 may be formed from polymer material, structural metal (e.g., structural steel), co-polymer material, thermoplastic and thermosetting polymers, resin-containing material, polyethylene, polystyrene, polypropylene, epoxy resins, phenolic resins, Acrylanitrile Butadiene Styrene (ABS), Polycarbonate (PC), Mix of ABS and PC, Acetal (POM), Acetate, Acrylic (PMMA), Liquid Crystal Polymer (LCP), Mylar, Polyamid-Nylon, Polyamid-Nylon 6, Polyamid-Nylon 11, Polybutylene Terephthalate (PBT), Polycarbonate (PC), Polyetherimide (PEI), Polyethylene (PE), Low Density PE (LDPE), High Density PE (HDPE), Ultra High Molecular Weight PE (UHMW PE), Polyethylene Terephthalate (PET), PolPolypropylene (PP), Polyphthalamide (PPA), Polyphenylenesulfide (PPS), Polystyrene (PS), High Impact Polystyrene (HIPS), Polysulfone (PSU), Polyurethane (PU), Polyvinyl Chloride (PVC), Chlorinated Polyvinyl chloride (CPVC), Polyvinylidenefluoride (PVDF), Styrene Acrylonitrile (SAN), Teflon TFE, Thermoplastic Elastomer (TPE), Thermoplastic Polyurethane (TPU), and/or Engineered Thermoplastic Polyurethane (ETPU), or any suitable combination thereof.

Attachment assembly 105 may be any suitable assembly for attaching assessment device 110 to a desired location. For example, attachment assembly 105 may include an attachment member 115 that may be received by assessment device 110 (e.g., by an aperture of assessment device 110). Attachment assembly 105 may also for example include a connecting member 120 that may connect attachment member 115 to an attachment member 125. For example, connecting member 120 may be a flexible member such as a cord, wire, or any other suitable flexible or inflexible (e.g., and/or elastic or inelastic) member. In at least some exemplary embodiments, connecting member 120 may be retractably received within attachment member 125. Attachment member 125 may be removably attached to clothing of a user and/or any other desired location via a member 130 (e.g., a clip, hook and loop fastener, magnetic member, and/or any other suitable member or fastener for attachment). In at least some exemplary embodiments, attachment assembly 105 may be a detachable lanyard attachment assembly and/or an employee badge clip. Attachment assembly 105 may thereby allow for assessment device 110 to be easily carried, stored, and/or utilized by a user. Assembly 100 may thereby be an easily portable assembly and assessment device.

Assessment device 110 may be formed from any suitable material for making a skin assessment. Assessment device 110 may include an attachment portion 135 for attaching assessment device 110 to attachment member 115 of attachment assembly 105. For example, attachment portion 135 may be an aperture that receives a portion of attachment member 115. Attachment portion 135 may be any other suitable attachment portion for attaching to a corresponding portion of attachment member 115 such as, for example, a magnetic component, a button, a clamp, a fastener such as a nut or screw, and/or any other suitable component for facilitating an attachment between assessment device 110 and attachment member 115.

Assessment device 110 may include a body member 140. In at least some exemplary embodiments, attachment portion 135 may be an aperture formed in body member 140. Body member 140 may be a substantially flat member having length and width dimensions that are significantly greater than a thickness of body member 140. For example, a length and/or width of body member 140 may be between about 10 and about 100 times as great as a thickness of body member 140. For example, body member 140 may have a length and a width dimension that are each between about 20 times and about 80 times larger than a thickness dimension of body member 140. Body member 140 may for example have a substantially constant thickness and a substantially flat surface running in a length and/or width direction on either side of body member 140. For example, body member 140 may be a sheet of material having a substantially constant thickness. For example, body member 140 may be a sheet member (e.g., a relatively thin member such as a relatively thin sheet member). Body member 140 may have, for example, a thickness of between about 1/32″ and about 1″, of between about 1/16″ and about ½″, or between about ⅛″ and about ¼″. The above exemplary relative dimensions may make body member 140 easily portable and allow a user to easily store and/or carry body member 140.

Body member 140 may be a transparent material (e.g., clear material) and may include material such as, for example, transparent plastic or glass. For example, body member 140 may include one or more of a glass polymer composite, a polycarbonate, fiberglass, glass, a fiber reinforced polymer, acrylic, and poly(methyl methacrylate) material. Body member 140 may include alkali-aluminosilicate material. For example, body member 140 may be an alkali-aluminosilicate sheet glass member. In at least some exemplary embodiments, body member 140 may include material such as Corning Gorilla® Glass. For example, body member 140 may include Plexiglas®, Lucite®, or Perspex® material. Body member 140 may be substantially entirely formed from acrylic and/or polycarbonate materials. Also for example, body member 140 may be formed from reusable plastic material or unbreakable glass material. Body member 140 may be formed, for example, from a transparent material that may be a see-through material, or a material having a property of being able to transmit rays of light through it so that elements disposed beyond the material can be clearly and distinctly seen.

Body member 140 may be formed from lightweight material. For example, body member 140 may be formed from material having a density of between about 0.75 g/cm³ (about 47 lb/ft³) and about 2.2 g/cm³ (about 137 lb/ft³), between about 0.8 g/cm³ (about 50 lb/ft³) and about 1.6 g/cm³ (about 100 lb/ft³), or between about 0.8 g/cm³ (about 50 lb/ft³) and about 1.2 g/cm³ (about 75 lb/ft³). The exemplary ranges may for example provide body member 140 having a desired strength and stiffness for applying pressure while also being lightweight and easily portable. For example, body member 140 may have a density that is greater than Polymethylpentene and less than PAI, 30% Glass Fiber.

Body member 140 may be a single, integral piece (e.g., single piece of material). For example, body member 140 may be formed as a single, integral piece of material formed from the exemplary materials disclosed herein.

Body member 140 may be scratch-resistant. For example, body member 140 may be formed from material such as material disclosed herein that is scratch-resistant. For example, a surface portion (e.g., surface) of body member 140 may have a scratch resistance as measured on the Mohs scale of mineral hardness of between about 5 and 10, between about 5 and about 9.8, between about 7 and about 9.5, or between about 7 and about 9.

Body member 140 may be nonporous. For example, body member 140 may be formed from material such as material disclosed herein that is nonporous. For example, body member 140 may have a porosity that is about equal to or less than Acrylonitrile butadiene styrene (ABS) plastic.

Body member 140 may include a plurality of markings 145. Markings 145 may be disposed along one, two, or more side portions (e.g., edges) of body member 140. Markings 145 may for example be a measurement scale that a user of assessment device 110 may use to assess (e.g., measure or track) a size of any suitable area of concern of a patient or other subject being evaluated. The measurement scale (e.g., distance between markings 145) may be of any suitable unit or scale such as, for example, centimeters, inches, millimeters, and/or any other desired unit or scale. Markings 145 may include any suitable indication or symbol such as tick marks, numbers, hash marks, and/or any other suitable marking that may denote units of length. Markings 145 may be disposed at a surface of body member 140 and/or may be disposed within (e.g., embedded within) an interior of body member 140. For example, markings 145 may be printed or disposed on a surface of body member 140 (e.g., flush with a surface of body member 140). Markings 145 may also be disposed within an interior of body member 140 so that they are visible to a user, but not at a surface of body member 140. For example, markings 145 may contain substantially no etchings and/or indentations (e.g., at a surface of body member 140) that may hold bacterial and/or viral residue. Markings 145 may be visible to a user and a surface at which or under which markings 145 are disposed may be smooth and/or flat so that markings 145 do not protrude above (e.g., or are not recessed within or below) a surface of body member 140. Accordingly for example, markings 145 may not have protrusions or recesses that interfere with an ease of cleaning (e.g., do not include recesses, indentations, protrusions, etchings, or other irregularities that may hold bacterial and/or viral residue). Users may thereby use antibacterial and antiviral wipes or any other suitable cleaning technique (e.g., cleaning material readily available in medical facilities or settings) to quickly and easily clean assembly 100. As described for example herein, body member 140 may have any desired configuration, shape, or size. Body member 140 may include rounded corners. For example, the exemplary disclosed body member may have any suitable shape such as a round-cornered triangle, round-cornered square, or a round-cornered rectangular shape. For example, body member 140 may have rounded corners or edges to avoid tissue damage from sharp edges.

Body member 140 may also have square, triangular, angled, and/or polygonal-shaped corners. Sides (e.g., edges) of body member 140 may be substantially straight, curved, and/or elliptically-shaped (e.g., round). In at least some exemplary embodiments, assessment device 110 may be configured to fit in a pocket such as a front shirt or pants pocket of a user so that it is easily portable, accessible, and quickly available for daily use. Assembly 100 including assessment device 110 may be sufficiently lightweight and slim so as to be easily carried in a pocket or other clothing component or accessory.

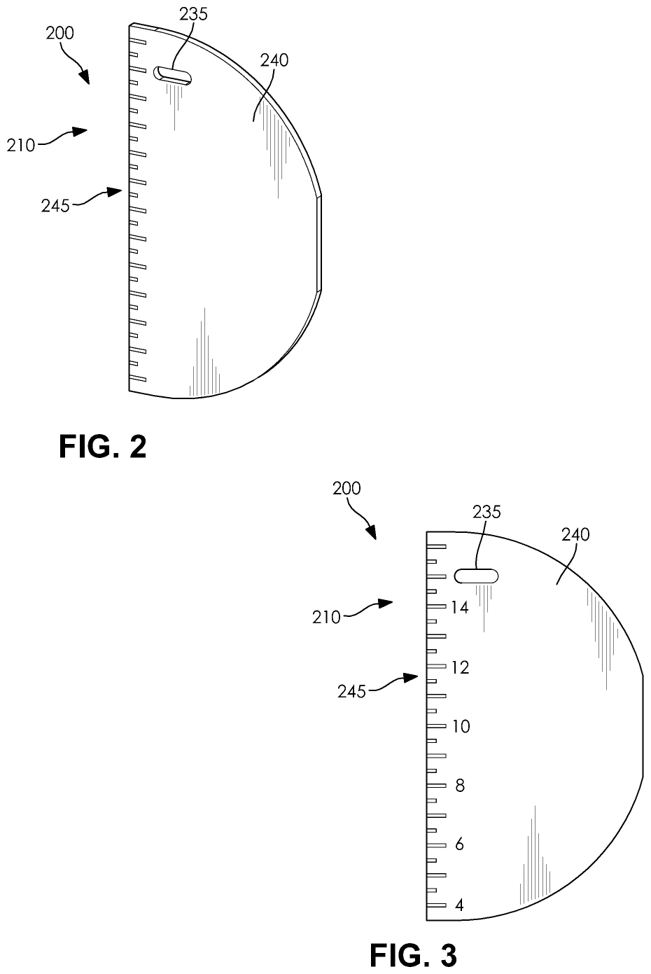

FIGS. 2 and 3 illustrate another exemplary embodiment of the exemplary disclosed assembly. Assembly 200 may include an assessment device 210 that may be generally similar to assessment device 110. Assessment device 210 may be attached to an attachment assembly that may be similar to attachment assembly 105. For example, assessment device 210 may have a body member 240 that may be generally similar to body member 140. Body member 240 may have an attachment portion 235 that may be generally similar to attachment portion 135 and a plurality of markings 245 that may be generally similar to markings 145. In at least some exemplary embodiments, attachment portion 235 may be an aperture that may receive a portion of the attachment assembly that may be similar to attachment assembly 105. Body member 240 may for example have a straight edge and a curved edge. Body member 240 may for example resemble an uppercase letter “D” in shape (e.g., have an uppercase letter “D” shape). The exemplary “D” shape may for example facilitate gripping by a user (e.g., on the curved edge) as the user places the straight edge at a desired location for assessing a wound or skin condition (e.g., rash or other area of concern).

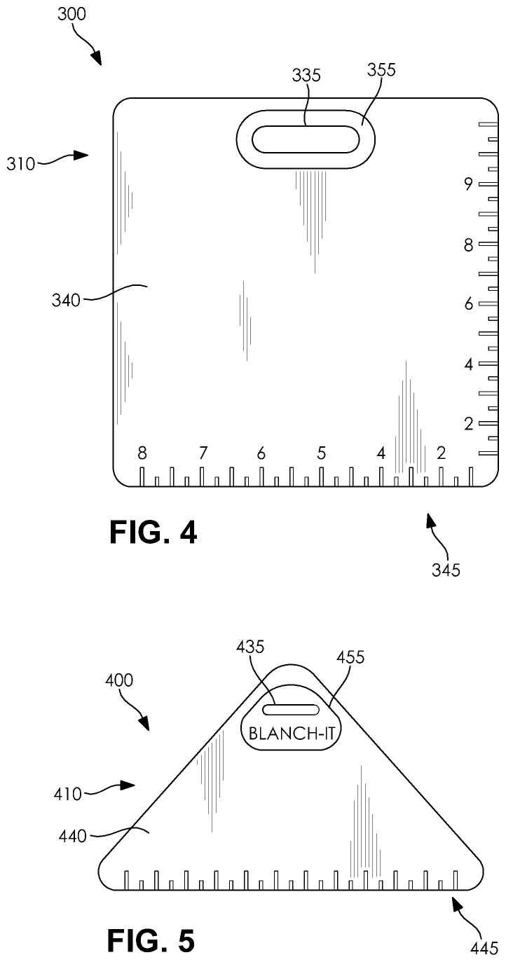

FIG. 4 illustrates another exemplary embodiment of the exemplary disclosed assembly. Assembly 300 may include an assessment device 310 that may be generally similar to assessment device 110. Assessment device 310 may be attached to an attachment assembly that may be similar to attachment assembly 105. For example, assessment device 310 may have a body member 340 that may be generally similar to body member 140. Body member 340 may have an attachment portion 335 that may be generally similar to attachment portion 135 and a plurality of markings 345 that may be generally similar to markings 145. Markings 345 may for example include a plurality of numbers for indicating units (e.g., inches, centimeters, and/or any other desired unit) of any suitable scale. In at least some exemplary embodiments, attachment portion 335 may be an aperture that may receive a portion of the attachment assembly that may be similar to attachment assembly 105. Body member 340 may be for example square, rectangular, polygonal, and/or any other suitable shape (e.g., having rounded or angular corners).

FIG. 5 illustrates another exemplary embodiment of the exemplary disclosed assembly. Assembly 400 may include an assessment device 410 that may be generally similar to assessment device 110. Assessment device 410 may be attached to an attachment assembly that may be similar to attachment assembly 105. For example, assessment device 410 may have a body member 440 that may be generally similar to body member 140. Body member 440 may have an attachment portion 435 that may be generally similar to attachment portion 135 and a plurality of markings 445 that may be generally similar to markings 145. Markings 445 may for example include a plurality of numbers for indicating units (e.g., inches, centimeters, and/or any other desired unit) of any suitable scale. In at least some exemplary embodiments, attachment portion 435 may be an aperture that may receive a portion of the attachment assembly that may be similar to attachment assembly 105. Body member 440 may be for example triangular (e.g., three-sided), polygonal, and/or any other suitable shape (e.g., having rounded or angular corners).

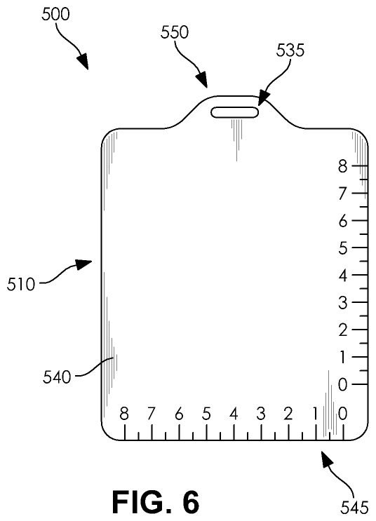

FIG. 6 illustrates another exemplary embodiment of the exemplary disclosed assembly. Assembly 500 may include an assessment device 510 that may be generally similar to assessment device 110. Assessment device 510 may be attached to an attachment assembly that may be similar to attachment assembly 105. For example, assessment device 510 may have a body member 540 that may be generally similar to body member 140. Body member 540 may have an attachment portion 535 that may be generally similar to attachment portion 135 and a plurality of markings 545 that may be generally similar to markings 145. Markings 545 may for example include a plurality of numbers for indicating units (e.g., inches, centimeters, and/or any other desired unit) of any suitable scale. In at least some exemplary embodiments, attachment portion 535 may be an aperture that may receive a portion of the attachment assembly that may be similar to attachment assembly 105. Body member 540 may be for example square, rectangular, triangular, polygonal, and/or any other suitable shape having rounded or angular corners. Body member 540 may include a protruding portion 550 that extends from a side portion of body member 540. Protruding portion 550 may for example include attachment portion 535.

In at least some exemplary embodiments, the exemplary disclosed apparatus may have a portion that may make the apparatus more visible to a user for example to avoid being lost or misplaced (e.g., mistakenly left on furniture such as on a desk or in a bed or other location). For example, assembly 300 may have a portion 355 and assembly 400 may have a portion 455 (e.g., and/or any of the other exemplary disclosed assemblies may have similar portions). Portions 355 and 455 may for example include coloring such as bright coloring (e.g., pink, red, yellow, neon color, or any other suitable noticeable color). The exemplary disclosed portions (e.g., portions 355 and/or 455) may be disposed at or around the exemplary attachment portions (e.g., attachment portions 335 and 435) or at any other desired portion of the exemplary disclosed assemblies (e.g., at edges, a central portion, or over an entire area of the exemplary disclosed body members). The exemplary disclosed portions (e.g., portions 355 and/or 455) may include or be a colored logo, thumb print, or other desired design. For example, exemplary disclosed portions (e.g., portions 355 and/or 455) may include or be a pink finger or thumbprint or any other desired design of any desired color. Similar to as described for example above regarding the exemplary markings (e.g., marking 145), the exemplary disclosed portions (e.g., portions 355 and/or 455) may be disposed flush with a flat surface of the exemplary disclosed skin assessment member or fully embedded within the exemplary disclosed skin assessment member. For example, the exemplary disclosed portions (e.g., portions 355 and/or 455) may be disposed flush with a surface or within the exemplary disclosed body member to make cleaning and disinfection easier.

In at least some exemplary embodiments, the exemplary disclosed apparatus may be a hand-held device that is a thin, single piece of clear, flat, scratch-resistant, nonporous, and reusable plastic or unbreakable glass material having a curved side and a straight side. The exemplary disclosed apparatus may be formed as an uppercase letter “D” in shape. The exemplary disclosed apparatus may have rounded corners to avoid inadvertent injury to both users and a patient or animal being evaluated.

In at least some exemplary embodiments, the exemplary disclosed apparatus may be a hand-held, reusable skin assessment and measurement tool that when pressed against the skin of a patient may be used to assess the patient for “non-blanchable” erythema, a skin condition (e.g., rash), and/or other injury. The exemplary disclosed apparatus may also assist users in the diagnosis, measurement, and subsequent management of tissue injury, rashes, and/or other areas of concern. The exemplary disclosed apparatus may be formed in one piece (e.g., a single, integral member) with no moving parts, which may be easily disinfected with antibacterial/antiviral wipes and may be small enough to carry or wear (e.g., wear on a user's clothing) for daily use.

In at least some exemplary embodiments, the exemplary disclosed apparatus may include a skin assessment member (e.g., body member 140). The skin assessment member may be transparent. The skin assessment member may be a single, integral member. The skin assessment member may be nonporous. A length and a width of the skin assessment member may be each between 20 times and 80 times larger than a thickness of the skin assessment member. A density of a material forming the skin assessment member may be between 0.75 g/cm³ and 2.2 g/cm³. The skin assessment member may include a plurality of markings (e.g., markings 245) that are disposed flush with a flat surface of the skin assessment member or fully embedded within the skin assessment member. The plurality of markings may include a plurality of numbers that are units of distance, the plurality of numbers fully embedded within the skin assessment member. The flat surface may be part of an exterior surface of the skin assessment member, and the exterior surface may be a completely smooth surface. A thickness of the skin assessment member may be between ⅛″ and 1/4″. The material forming the skin assessment member may include plastic or glass. The skin assessment member may include an aperture configured to receive a portion of an attachment assembly (e.g., attachment assembly 105). The skin assessment member may be scratch-resistant. The skin assessment member may have a scratch resistance as measured on the Mohs scale of mineral hardness of between 7 and 9. The skin assessment member may have a curved edge and a straight edge. The skin assessment member may have an uppercase letter “D” shape.

In at least some exemplary embodiments, the exemplary disclosed apparatus may include a skin assessment member (e.g., body member 140). The skin assessment member may be transparent. The skin assessment member may be a single, integral member. The skin assessment member may have a porosity that is equal to or less than Acrylonitrile butadiene styrene plastic. A length and a width of the skin assessment member may each be between 20 times and 80 times larger than a thickness of the skin assessment member. A density of a material forming the skin assessment member may be between 0.8 g/cm³ and 1.6 g/cm³. The skin assessment member may be scratch-resistant. The skin assessment member may include a plurality of markings (e.g., markings 145) that are fully embedded within the skin assessment member. A thickness of the skin assessment member may be between ⅛″ and 1/4″. The skin assessment member may include an aperture (e.g., attachment portion 135) configured to receive a portion of a lanyard assembly. The skin assessment member may have a scratch resistance as measured on the Mohs scale of mineral hardness of between 7 and 9. The skin assessment member may have a curved edge and a straight edge. The skin assessment member may have an uppercase letter “D” shape.

The exemplary disclosed apparatus and method may be used in any suitable application for making a skin assessment. For example, the exemplary disclosed apparatus and method may be used in any application involving assessing skin for human patients, animals (e.g., for veterinarian applications), and/or any other suitable application involving assessing skin. Also for example, the exemplary disclosed apparatus and method may be used in any suitable location or activity for making skin assessments such as medical facilities, elder care facilities, residences, operational locations such as military or law enforcement operations areas, commercial or residential locations, and/or any other suitable locations.

In at least some exemplary embodiments, the exemplary disclosed apparatus may be a skin assessment diascope and measurement tool for rapidly assessing, measuring, and/or differentiating tissue damage such as Stage 1 pressure ulcers and/or non-blanching or petechial skin rashes that may occur with Meningitis, Sepsis, and/or Henoch-Schonlein purpura. The exemplary disclosed apparatus may also be used to detect and measure any other suitable skin damage such as deep tissue injuries.

In at least some exemplary embodiments, the exemplary disclosed apparatus and method may be used in evaluating non-blanchable erythema and/or rashes that may be an indicator in some or all age groups of existing tissue injury or life-threatening illness. The exemplary disclosed apparatus may be a tool that rapidly assesses, measures, and/or differentiates tissue damage and skin rashes, by a response of tissue evaluated by the exemplary apparatus to blanching. The exemplary disclosed apparatus and method may be used in both a medical setting as well as by trained laypeople to evaluate (e.g., define) tissue injury and also to prevent further damage or progression of serious illness.

An exemplary operation of the exemplary disclosed apparatus and method will now be described. A user (e.g., practitioner, caregiver, or any other suitable user) may clean all surfaces of the exemplary disclosed assembly with appropriate antibacterial and antiviral solution or wipes (e.g., or other cleaning material) both before and after use (e.g., both pre- and post-assessment). When the exemplary disclosed assembly is dry, the user applies a flat surface of the assembly against the skin to be observed (e.g., against skin of patient to be assessed), applying suitable pressure to “blanch” the surrounding tissue (e.g., tissue surrounding patient's wound). For example, the user may press the exemplary disclosed assembly against the patient's skin to blanch the skin, observing the blanching through the transparent or clear material of the exemplary disclosed assembly. The user may observe (e.g., through the transparent material) color changes in the patient's skin as pressure is applied, maintained, and/or removed. The user may apply limited but constant pressure to the skin area of concern using the exemplary disclosed assembly.

If the skin of concern is “non-blanchable,” a user who is a practitioner in a hospital, clinic, or other medical facility would follow best practice protocols to prevent further skin breakdown. If a rash is non-blanchable, the practitioner would without delay screen the patient further for more serious disease processes. If the skin of concern is “non-blanchable” in non-medical settings, the user would seek medical follow-up (e.g., activate emergency medical services or take any other suitable action). Such assessment (e.g., measurement) of the skin areas of concern may be completed immediately (e.g., simultaneously with other actions) rather than a user leaving the patient to find other measuring instruments.

The user may make any suitable medical determinations based on the objective evidence provided by use of the exemplary disclosed assembly. The user may evaluate “blanchable” and/or “non-blanchable” erythema. For example if the erythematous area blanches (e.g., turns white), objective evidence that the patient's microcirculation is intact is observed and/or noted by the user. If the erythematous area does not blanch, evidence that microcirculatory damage exists is noted by the user (e.g., based on the intensity or duration of the pressure). The user may utilize the objective evidence provided by use of the exemplary disclosed assembly in any useful manner based on his or her experience and training.

For example, the user may determine that a stage 1 pressure injury (e.g., non-blanchable erythema of intact skin) exists based on objective evidence provided by the exemplary disclosed assembly. For example, use of the exemplary disclosed assembly may provide objective evidence that a skin area of concern is intact with a localized area of non-blanchable erythema.

Also for example, the user may determine that a deep tissue pressure injury (e.g., persistent non-blanchable, purple, deep red, or maroon discoloration) exists based on objective evidence provided by the exemplary disclosed assembly. For example, use of the exemplary disclosed assembly may provide objective evidence that a skin area of concern is intact or non-intact with a localized area of persistent, non-blanchable deep red, purple, or maroon discoloration and/or epidermal separation.

In at least some exemplary embodiments, the exemplary disclosed method may include providing a medical device including a skin assessment member (e.g., body member 140). The skin assessment member may be transparent. The skin assessment member may be a single, integral member. The skin assessment member may be nonporous. The skin assessment member may include a plurality of markings (e.g., markings 145) that are disposed flush with a flat surface of the skin assessment member or fully embedded within the skin assessment member. The flat surface may be part of a smooth exterior surface of the skin assessment member. The exemplary disclosed method may also include cleaning the smooth exterior surface and applying constant pressure with the skin assessment member against a skin area of a patient. The plurality of markings may be disposed over a wound or rash of the skin area. A length and a width of the skin assessment member may each be between 20 times and 80 times larger than a thickness of the skin assessment member. A density of a material forming the skin assessment member may be between 0.8 g/cm³ and 1.2 g/cm³. Applying constant pressure may include holding a curved edge of the skin assessment member that has an uppercase letter “D” shape. The skin assessment member may have a porosity that is equal to or less than Acrylonitrile butadiene styrene plastic.

The exemplary disclosed method and apparatus may provide an effective technique for making a skin assessment of a human patient. For example, the exemplary disclosed method and apparatus may provide a cost-effective and easily trainable device and technique for making a skin assessment. Also for example, the exemplary disclosed method and apparatus may provide a device that may be easily cleaned and decontaminated. The exemplary disclosed method and apparatus may provide an efficient method for making objective skin assessments. The exemplary disclosed method and apparatus may facilitate effective analysis (e.g., human eye analysis) by a user by applying limited but constant pressure to tissue being assessed. The exemplary disclosed method and apparatus may provide objective results that are easily viewed and stable based on use of the exemplary assessment device. The exemplary disclosed method and apparatus may provide efficient assessment of a patient having poor mobility, where more than one caregiver may be involved in efforts to suitably turn or position the patient. The exemplary disclosed method and apparatus may provide a readily available measuring device that one user may effectively utilize to assess and track tissue damage as soon as the condition is detected. The exemplary disclosed method and apparatus may avoid situations in which caregivers leave a patient unattended in order to find disposable measuring tools and/or find co-workers or family members to move that patient for measurement.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as described herein-above may be used. Many suitable materials are known and available and can be selected and mixed depending on desired strength and flexibility, preferred manufacturing method and particular use, as will be apparent to a person of ordinary skill in the art.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. An apparatus, comprising:
a skin assessment member;
wherein the skin assessment member is transparent;
wherein the skin assessment member is a single, integral member;
wherein the skin assessment member is nonporous;
wherein a length and a width of the skin assessment member are each between 20 times and 80 times larger than a thickness of the skin assessment member;
wherein a density of a material forming the skin assessment member is between 0.75 g/cm³ and 2.2 g/cm³; and
wherein the skin assessment member includes a plurality of markings that are disposed flush with a flat surface of the skin assessment member or fully embedded within the skin assessment member.

2. The apparatus of claim 1, wherein the plurality of markings include a plurality of numbers that are units of distance, the plurality of numbers being fully embedded within the skin assessment member.

3. The apparatus of claim 1, wherein the flat surface is part of an exterior surface of the skin assessment member, and the exterior surface is a completely smooth surface.

4. The apparatus of claim 1, wherein a thickness of the skin assessment member is between ⅛″ and ¼″.

5. The apparatus of claim 1, wherein the material forming the skin assessment member includes plastic or glass.

6. The apparatus of claim 1, wherein the skin assessment member includes an aperture configured to receive a portion of an attachment assembly.

7. The apparatus of claim 1, wherein the skin assessment member is scratch-resistant.

8. The apparatus of claim 7, wherein the skin assessment member has a scratch resistance as measured on the Mohs scale of mineral hardness of between 7 and 9.

9. The apparatus of claim 1, wherein the skin assessment member has a curved edge and a straight edge.

10. The apparatus of claim 9, wherein the skin assessment member has an uppercase letter “D” shape.

11. A method, comprising:
providing an apparatus including a skin assessment member;
wherein the skin assessment member is transparent;
wherein the skin assessment member is a single, integral member;
wherein the skin assessment member is nonporous;
wherein the skin assessment member includes a plurality of markings that are disposed flush with a flat surface of the skin assessment member or fully embedded within the skin assessment member; and
wherein the flat surface is part of a smooth exterior surface of the skin assessment member;
cleaning the smooth exterior surface; and
applying constant pressure with the skin assessment member against a skin area of a patient;
wherein the plurality of markings are disposed over a wound or a skin condition of the skin area.

12. The method of claim 11, wherein a length and a width of the skin assessment member are each between 20 times and 80 times larger than a thickness of the skin assessment member.

13. The method of claim 11, wherein a density of a material forming the skin assessment member is between 0.8 g/cm³ and 1.2 g/cm³.

14. The method of claim 11, wherein applying constant pressure includes holding a curved edge of the skin assessment member that has an uppercase letter “D” shape.

15. The method of claim 11, wherein the skin assessment member has a porosity that is equal to or less than Acrylonitrile butadiene styrene plastic.

16. An apparatus, comprising:
a skin assessment member;
wherein the skin assessment member is transparent;
wherein the skin assessment member is a single, integral member;
wherein the skin assessment member has a porosity that is equal to or less than Acrylonitrile butadiene styrene plastic;
wherein a length and a width of the skin assessment member are each between 20 times and 80 times larger than a thickness of the skin assessment member;
wherein a density of a material forming the skin assessment member is between 0.8 g/cm³ and 1.6 g/cm³;
wherein the skin assessment member is scratch-resistant;
wherein the skin assessment member includes a plurality of markings that are fully embedded within the skin assessment member; and
wherein a thickness of the skin assessment member is between ⅛″ and ¼″.

17. The apparatus of claim 16, wherein the skin assessment member includes an aperture configured to receive a portion of a lanyard assembly.

18. The apparatus of claim 16, wherein the skin assessment member has a scratch resistance as measured on the Mohs scale of mineral hardness of between 7 and 9.

19. The apparatus of claim 16, wherein the skin assessment member has a curved edge and a straight edge.

20. The apparatus of claim 19, wherein the skin assessment member has an uppercase letter “D” shape.
